(19) United States
(12) Patent Application Publication
(10) Pub. No.: US 2026/0120885 A1
(43) Pub. Date: Apr. 30, 2026

(54) METHOD AND APPARATUS FOR PREDICTING PAIN-SCORE USING PRE-LEARNED ARTIFICIAL INTELLIGENCE MODEL

(71) Applicant: Seoul National University Hospital, Seoul (KR)

(72) Inventors: Insun PARK, Seoul (KR); Jae Hyon PARK, Seoul (KR)

(21) Appl. No.: 19/333,797

(22) Filed: Sep. 19, 2025

(30) Foreign Application Priority Data
Sep. 20, 2024 (KR) 10-2024-0127702

(51) Int. Cl. G16H 50/30; G06V 40/16; G16H 30/40

(52) U.S. Cl. CPC G16H 50/30; G06V 40/171; G16H 30/40

(57) ABSTRACT

There is provided a method for predicting a pain score using a pre-trained artificial intelligence model, the method comprising: extracting features for at least one action unit (AU) representing a facial expression by inputting a facial image of a target patient; and determining the pain score of the target patient by inputting the features for the at least one AU representing the facial expression into the pre-trained artificial intelligence model, wherein the artificial intelligence model is trained to determine the pain score based on a first weight assigned to an AU associated with a patient's lip shape.

### CROSS-REFERENCE TO RELATED APPLICATIONS

The present application claims priority to Korean Patent Application No. 10-2024-0127702, filed Sep. 20, 2024, the entire contents of which are incorporated here for all purposes by this reference.

### TECHNICAL FIELD

Embodiments of the present disclosure relate to a method and apparatus for predicting a pain score using a pre-trained artificial intelligence model.

### BACKGROUND

Pain is a subjective experience reported by a patient, making it difficult to accurately assess. Although subjective pain assessment is performed using the patient-reported numeric pain scale (Numeric Rating Scale, NRS), this method requires the patient to personally state their pain intensity on a scale of 0 to 10.

Herein, while guidelines are provided for determining pain intensity on the scale of 0 to 10, not only may the standard be considered different for each patient, but there is also a limitation that pain assessment for patients with communication difficulties cannot be adequately performed.

To overcome this limitation, various attempts have been made to objectively assess pain. Recently, a pain assessment method based on the Analgesia Nociception Index (ANI), which is determined by analyzing ECG or EKG waveforms, is being utilized.

However, the pain assessment method based on the Analgesia Nociception Index has a problem of low pain prediction accuracy for acute pain patients or post-operative pain patients.

Accordingly, there is a need to develop a technology that objectively assesses a target patient's pain regardless of the patient's type, by determining the target patient's pain score using a pre-trained artificial intelligence model based on features related to facial expressions.

### SUMMARY

An object of an embodiment is to address a problem by objectively assessing the pain of patients with communication difficulties, such as post-operative patients, children, the elderly, and psychiatric patients with dementia or severe depression, by automatically recognizing facial expressions to interpret pain.

Furthermore, an object of an embodiment is to address a problem by more accurately assessing the pain of patients using an artificial intelligence model trained to determine a pain score based on a weight assigned to an AU associated with the patient's lip shape.

In accordance with an aspect of the present disclosure, there is provided a method for predicting a pain score using a pre-trained artificial intelligence model, the method comprising: extracting features for at least one action unit (AU) representing a facial expression by inputting a facial image of a target patient; and determining the pain score of the target patient by inputting the features for the at least one AU representing the facial expression into the pre-trained artificial intelligence model, wherein the artificial intelligence model is trained to determine the pain score based on a first weight assigned to an AU associated with a patient's lip shape.

In the extracting of the features for the at least one AU, the target patient is an acute pain patient or a post-operative pain patient.

The extracting of the features for the at least one AU comprises: determining a facial feature point of the target patient using an OpenFace algorithm, and extracting the features for the at least one AU representing the facial expression based on matching the facial feature point of the target patient and an AU set.

The extracting of the features for the at least one AU further comprises: extracting features for a gaze or a facial direction of the target patient.

The determining of the pain score comprises: determining the pain score by further inputting features related to a gaze or a facial direction of the target patient into the pre-trained artificial intelligence model.

The artificial intelligence model is trained to determine the pain score based on a training dataset including a facial image of the patient and a Numeric Rating Scale (NRS) label corresponding to the pain of the patient.

The artificial intelligence model is further trained to determine the pain score based on a second weight assigned to features related to a gaze of the patient or a third weight assigned to features related to a facial direction of the patient.

The first weight is higher than the second weight and the third weight.

In accordance with an aspect of the present disclosure, there is provided an apparatus for predicting a pain score using a pre-trained artificial intelligence model, the apparatus comprising: a memory storing a pain score prediction program including one or more instructions; and a processor that loads the pain score prediction program from the memory and executes the pain score prediction program, wherein the one or more instructions, when executed by the processor, cause the processor to: input a facial image of a target patient to extract features for at least one action unit (AU) representing a facial expression, and input the features for the at least one AU representing the facial expression into the pre-trained artificial intelligence model to determine the pain score of the target patient, wherein the artificial intelligence model is trained to determine the pain score based on a first weight assigned to an AU associated with a patient's lip shape.

The target patient is an acute pain patient or a post-operative pain patient.

The one or more instructions, when executed by the processor, cause the processor to: determine a facial feature point of the target patient using an OpenFace algorithm, and extract the features for the at least one AU representing the facial expression based on matching the facial feature point of the target patient and an AU set.

The one or more instructions, when executed by the processor, cause the processor to extract features for a gaze or a facial direction of the target patient.

The one or more instructions, when executed by the processor, cause the processor to determine the pain score by further inputting features related to a gaze or a facial direction of the target patient into the pre-trained artificial intelligence model.

The artificial intelligence model is trained to determine the pain score based on a training dataset including a facial image of the patient and a Numeric Rating Scale (NRS) label corresponding to the pain of the patient.

The artificial intelligence model is further trained to determine the pain score based on a second weight assigned to features related to a gaze of the patient or a third weight assigned to features related to a facial direction of the patient.

The first weight is higher than the second weight and the third weight.

In accordance with an aspect of the present disclosure, there is provided a computer-readable recording medium storing a computer program, wherein the computer program, when executed by a processor, causes the processor to: extract features for at least one action unit (AU) representing a facial expression by inputting a facial image of a target patient; and determine the pain score of the target patient by inputting the features for the at least one AU representing a facial expression into a pre-trained artificial intelligence model, wherein the artificial intelligence model is trained to determine the pain score based on a first weight assigned to an AU associated with a patient's lip shape.

In accordance with an aspect of the present disclosure, there is provided a computer program stored on a computer-readable recording medium, wherein the computer program, when executed by a processor, causes the processor to: extract features for at least one action unit (AU) representing a facial expression by inputting a facial image of a target patient; and determine the pain score of the target patient by inputting the features for the at least one AU representing a facial expression into a pre-trained artificial intelligence model, wherein the artificial intelligence model is trained to determine the pain score based on a first weight assigned to an AU associated with a patient's lip shape.

According to an embodiment, when the target patient is an acute pain patient or a post-operative pain patient, the pain score prediction accuracy of the artificial intelligence model may be improved as the model is trained based on a weight on AUs associated with lip shape.

Furthermore, according to an embodiment, by determining a pain score using a pre-trained artificial intelligence model based on features for an AU representing a facial expression, it is possible not only to automatically recognize facial expressions to interpret the target patient's pain, but also to objectively and accurately assess the pain of patients with communication difficulties, such as post-operative patients, children, the elderly, and psychiatric patients with dementia or severe depression.

Furthermore, according to an embodiment, by objectively and accurately determining the pain of patients, pain management for the patient may be performed promptly and appropriately.

Furthermore, according to an embodiment, it becomes possible to reduce the continuous fatigue of medical staff for pain assessment and medical costs.

### BRIEF DESCRIPTION OF THE DRAWINGS

FIG. 1 is a block diagram illustrating a pain score prediction apparatus according to an embodiment.

FIG. 2 is a block diagram conceptually illustrating the functions of a pain score prediction program according to an embodiment.

FIG. 3 is a flowchart illustrating a pain score prediction method according to an embodiment.

FIG. 4 is a diagram exemplarily illustrating at least one AU according to an embodiment.

FIG. 5 is a diagram exemplarily illustrating the extraction of features for a target patient's gaze or facial direction and features for at least one AU according to an embodiment.

FIG. 6 is a diagram exemplarily illustrating the pain score prediction accuracy determined based on at least one AU representing a facial expression according to an embodiment.

FIG. 7 is a diagram exemplarily illustrating a training dataset of an artificial intelligence model according to an embodiment.

### DETAILED DESCRIPTION OF THE INVENTION

The advantages and features of the embodiments and the methods of accomplishing the embodiments will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

Terms used in the present specification will be briefly described, and the present disclosure will be described in detail.

In terms used in the present disclosure, general terms currently as widely used as possible while considering functions in the present disclosure are used. However, the terms may vary according to the intention or precedent of a technician working in the field, the emergence of new technologies, and the like. In addition, in certain cases, there are terms arbitrarily selected by the applicant, and in this case, the meaning of the terms will be described in detail in the description of the corresponding invention. Therefore, the terms used in the present disclosure should be defined based on the meaning of the terms and the overall contents of the present disclosure, not just the name of the terms.

When it is described that a part in the overall specification “includes” a certain component, this means that other components may be further included instead of excluding other components unless specifically stated to the contrary.

In addition, a term such as a “unit” or a “portion” used in the specification means a software component or a hardware component such as FPGA or ASIC, and the “unit” or the “portion” performs a certain role. However, the “unit” or the “portion” is not limited to software or hardware. The “portion” or the “unit” may be configured to be in an addressable storage medium, or may be configured to reproduce one or more processors. Thus, as an example, the “unit” or the “portion” includes components (such as software components, object-oriented software components, class components, and task components), processes, functions, properties, procedures, subroutines, segments of program code, drivers, firmware, microcode, circuits, data, database, data structures, tables, arrays, and variables. The functions provided in the components and “unit” may be combined into a smaller number of components and “units” or may be further divided into additional components and “units”.

Hereinafter, the embodiment of the present disclosure will be described in detail with reference to the accompanying drawings so that those of ordinary skill in the art may easily implement the present disclosure. In the drawings, portions not related to the description are omitted in order to clearly describe the present disclosure.

Hereinafter, embodiments of the present disclosure will be described in detail with reference to the attached drawings.

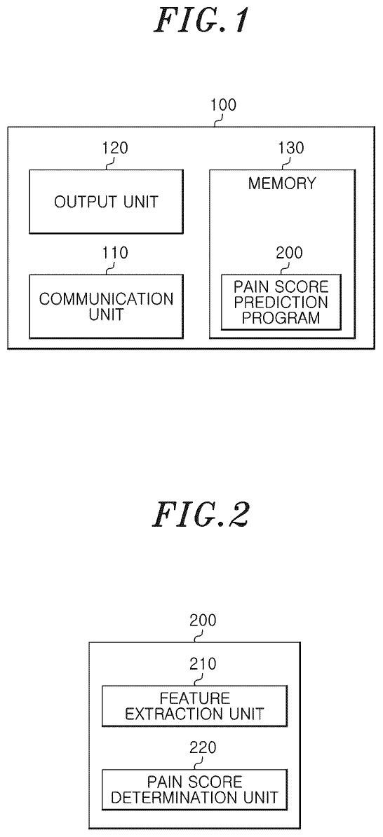

FIG. 1 is a block diagram illustrating a pain score prediction apparatus according to an embodiment.

Referring to FIG. 1, the pain score prediction apparatus 100 may include a processor 110, an input/output device 120, and a memory 130.

The processor 110 may control the overall operation of the pain score prediction apparatus 100.

The processor 110 may receive a facial image of a target patient using the input/output device 120. Herein, the facial image may refer to a frontal or side image of the target patient captured by a camera.

In an embodiment, it is described that the facial image of the target patient is input through the input/output device 120, but the disclosure is not limited thereto. That is, according to an embodiment, the pain score prediction apparatus 100 may include a transceiver (not shown), and the pain score prediction apparatus 100 may also receive the facial image of the target patient using the transceiver (not shown), or the facial image of the target patient may be generated within the pain score prediction apparatus 100.

The processor 110 may input a facial image of a target patient to extract features for at least one action unit (AU) representing a facial expression, and input the features for the at least one AU representing the facial expression into a pre-trained artificial intelligence model to determine the pain score of the target patient.

The input/output device 120 may include one or more input devices and/or one or more output devices. For example, the input device may include a microphone, keyboard, mouse, touch screen, etc., and the output device may include a display, speaker, etc.

The memory 130 may store a pain score prediction program 200 and information necessary for executing the pain score prediction program 200.

In this specification, the pain score prediction program 200 may refer to software including instructions for determining a target patient's pain score using a pre-trained artificial intelligence model.

The processor 110 may load the pain score prediction program 200 and information necessary for executing the pain score prediction program 200 from the memory 130 in order to execute the pain score prediction program 200.

The processor 110 may execute the pain score prediction program 200 to input features for at least one AU representing a facial expression and determine a pain score.

The functions and/or operations of the pain score prediction program 200 will be described in detail with reference to FIG. 2.

FIG. 2 is a block diagram conceptually illustrating the functions of a pain score prediction program according to an embodiment.

Referring to FIG. 2, the pain score prediction program 200 may include a feature extraction unit 210 and a pain score determination unit 220.

The feature extraction unit 210 and the pain score determination unit 220 shown in FIG. 2 are conceptual divisions of the functions of the pain score prediction program 200 for ease of explanation and are not limited thereto. According to embodiments, the functions of the feature extraction unit 210 and the pain score determination unit 220 may be merged or separated, and may be implemented as a series of instructions included in a single program.

First, the feature extraction unit 210 may input a facial image of a target patient to extract features for at least one action unit (AU) representing a facial expression.

Herein, the target patient may refer to an acute pain patient or a post-operative pain patient, and not a chronic pain patient.

Furthermore, the at least one AU may be a unit of facial movement determined through facial components (e.g., eyebrows, eyes, nose, mouth, etc.) and may be included in an AU set (e.g., AU1 to AU64) of the Facial Action Coding System (FACS), which is a method for describing facial expressions through the movement of muscles in the human face based on anatomy.

The at least one AU according to an embodiment may include an AU corresponding to an upper facial region, a lower facial region, a facial direction, or a gaze. Specifically, the at least one AU may include AU1 to AU7 and AU41 to AU46 corresponding to the upper facial region, AU9 to AU28 corresponding to the lower facial region, AU51 to AU58 corresponding to the facial direction, and AU61 to AU64 corresponding to the gaze.

Herein, the features for the at least one AU may refer to a binarized value (e.g., 0 or 1) that determines whether the facial image corresponds to an AU corresponding to the upper facial region, the lower facial region, the facial direction, or the gaze, or a value related to the intensity of the corresponding AU.

According to an embodiment, the feature extraction unit 210 may determine a facial feature point of the target patient using an OpenFace algorithm.

Herein, the OpenFace algorithm may refer to an algorithm that recognizes facial feature points by detecting facial landmarks, aligning the face, and then extracting a feature vector.

Furthermore, the feature extraction unit 210 may extract features for at least one AU representing a facial expression based on matching a facial feature point of the target patient and an AU set.

In one embodiment, the feature extraction unit 210 may extract features for an AU associated with the target patient's lip shape (e.g., AU10 to AU18, AU20, and AU22 to AU28) based on the matching.

In another embodiment, the feature extraction unit 210 may extract features for an AU associated with the target patient's facial direction (e.g., AU51 to AU58) based on the matching.

In another embodiment, the feature extraction unit 210 may extract features for an AU associated with the target patient's gaze (e.g., AU61 to AU64) based on the matching.

According to another embodiment, the feature extraction unit 210 may extract features for the target patient's gaze or facial direction regardless of the matching. Specifically, the feature extraction unit 210 may extract features for the target patient's gaze or facial direction from the target patient's facial image using the OpenFace algorithm.

Meanwhile, the algorithm used to extract the features for the at least one AU and the features for the target patient's gaze or facial direction is merely an example and is not limited thereto, and may be variously changed within a range to achieve the object of the disclosure.

Next, the pain score determination unit 220 may input the features for the at least one AU representing a facial expression into a pre-trained artificial intelligence model to determine the pain score of the target patient.

The artificial intelligence model according to an embodiment may be one that is trained to determine a pain score based on a training dataset that includes patients'facial images and corresponding Numeric Rating Scale (NRS) labels for the patients'pain.

Herein, the NRS corresponding to the patient's pain may refer to a discrete pain score having a range of 0 to 10 as a patient-reported numeric pain scale. An NRS label according to an embodiment may refer to a multi-label that classifies a patient's facial image from 0 to 10 based on an assessment by medical staff.

Meanwhile, since analgesics (e.g., fentanyl) should be administered to a patient by medical staff when the NRS is 7 or more, an NRS of 7 may be considered a significant value.

Referring to this, an NRS label according to an embodiment may refer to a binarized label that classifies a patient's facial image as 0 (i.e., when NRS is less than 7) or 1 (i.e., when NRS is 7 or more) based on an assessment by medical staff.

Specifically, the artificial intelligence model may be trained to determine the pain score based on a first weight assigned to an AU associated with the patient's lip shape.

In one embodiment, the artificial intelligence model may be trained to determine a pain score by inputting features for at least one AU, based on weights assigned to AU10 to AU18, AU20, and AU22 to AU28.

When the target patient is an acute pain patient or a post-operative pain patient, a unique effect of improving pain score prediction accuracy may be achieved by performing inference using an artificial intelligence model trained by placing a weight on AUs associated with lip shape.

Furthermore, the pain score determination unit 220 may further input features related to the target patient's gaze or facial direction into the pre-trained artificial intelligence model to determine the pain score of the target patient.

The artificial intelligence model according to an embodiment may be further trained to determine the pain score based on a second weight assigned to features related to the patient's gaze or a third weight assigned to features related to the patient's facial direction.

Herein, the second weight may refer to a weight assigned to AU61 to AU64 associated with the patient's gaze or a weight assigned to features for the gaze directly extracted from the target patient's facial image.

Furthermore, the third weight may refer to a weight assigned to AU51 to AU58 associated with the patient's facial direction or a weight assigned to features for the facial direction directly extracted from the target patient's facial image.

Meanwhile, during the training process of the artificial intelligence model, the first weight may be set higher than the second weight and the third weight.

As such, by determining a pain score using a pre-trained artificial intelligence model, it is possible to automatically recognize facial expressions to interpret the target patient's pain, and a unique effect may be achieved in that the pain of patients with communication difficulties, such as post-operative patients, children, the elderly, and psychiatric patients with dementia or severe depression, may be objectively and accurately assessed.

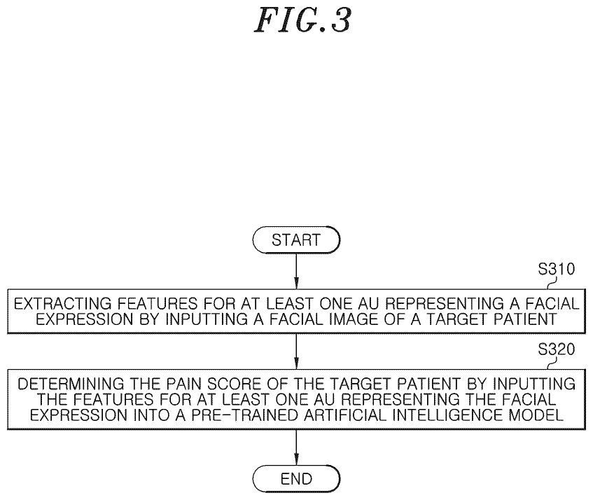

FIG. 3 is a flowchart illustrating a pain score prediction method according to an embodiment.

Referring to FIG. 2 and FIG. 3, the feature extraction unit 210 may input a facial image of a target patient to extract features for at least one AU representing a facial expression (S310).

Then, the pain score determination unit 220 may input the features for the at least one AU representing the facial expression into a pre-trained artificial intelligence model to determine the pain score of the target patient (S320). Herein, the artificial intelligence model may be one that is trained to determine the pain score based on a first weight assigned to an AU associated with a patient's lip shape.

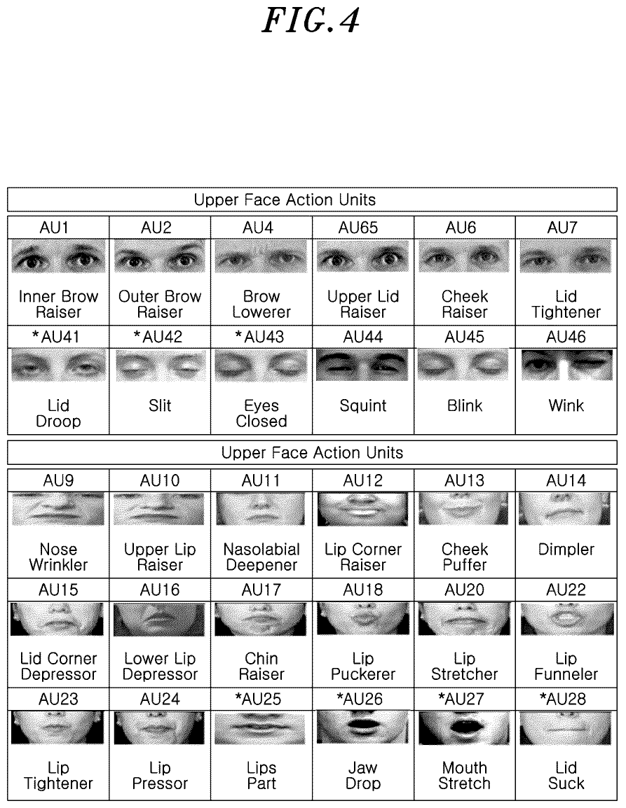

FIG. 4 is a diagram exemplarily illustrating at least one AU according to an embodiment.

FIG. 4 illustrates an AU corresponding to an upper facial region and an AU corresponding to a lower facial region.

Specifically, the at least one AU may include AU1 to AU7 and AU41 to AU46 corresponding to the upper facial region and AU9 to AU28 corresponding to the lower facial region.

For example, AU4 is ‘Brow Lowerer’, which may refer to a facial movement unit related to moving the eyebrows downward.

Furthermore, for example, AU24 is ‘Lip Pressor’, which may refer to a facial movement unit related to tensing the lips.

Meanwhile, the at least one AU may further include AU51 to AU58 corresponding to facial direction, and AU61 to AU64 corresponding to gaze.

For example, AU53 is ‘Head Up’, which may refer to a facial movement unit related to lifting the head.

Furthermore, for example, AU62 is ‘Eyes Turn Right’, which may refer to a facial movement unit related to moving the eyes to the right.

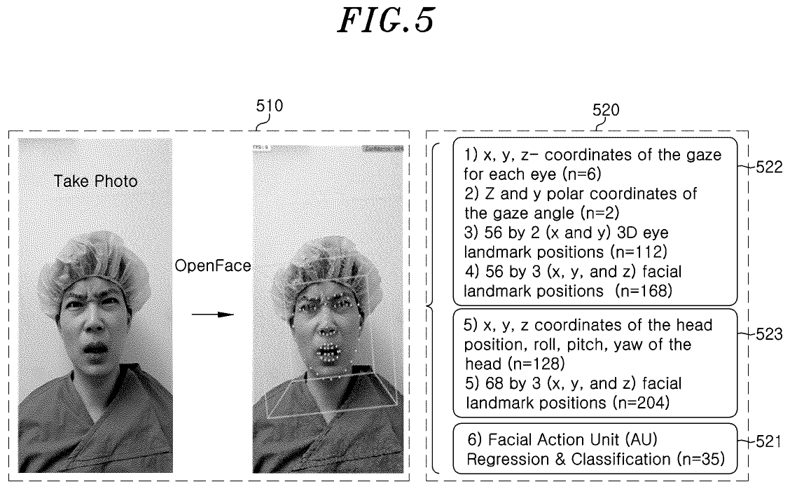

FIG. 5 is a diagram exemplarily illustrating the extraction of features for a target patient's gaze or facial direction and features for at least one AU according to an embodiment.

Referring to FIG. 2 and FIG. 5, the feature extraction unit 210 may determine the facial feature points of the target patient from the target patient's facial image using the OpenFace algorithm 510.

Then, the feature extraction unit 210 may extract features from the facial feature points for input to the pre-trained artificial intelligence model 520.

Specifically, the feature extraction unit 210 may extract features for at least one AU representing a facial expression 521 based on matching the target patient's facial feature points and an AU set.

Furthermore, the feature extraction unit 210 may extract features for the target patient's gaze 522 from the target patient's facial feature points.

Furthermore, the feature extraction unit 210 may extract features for the target patient's facial direction 523 from the target patient's facial feature points.

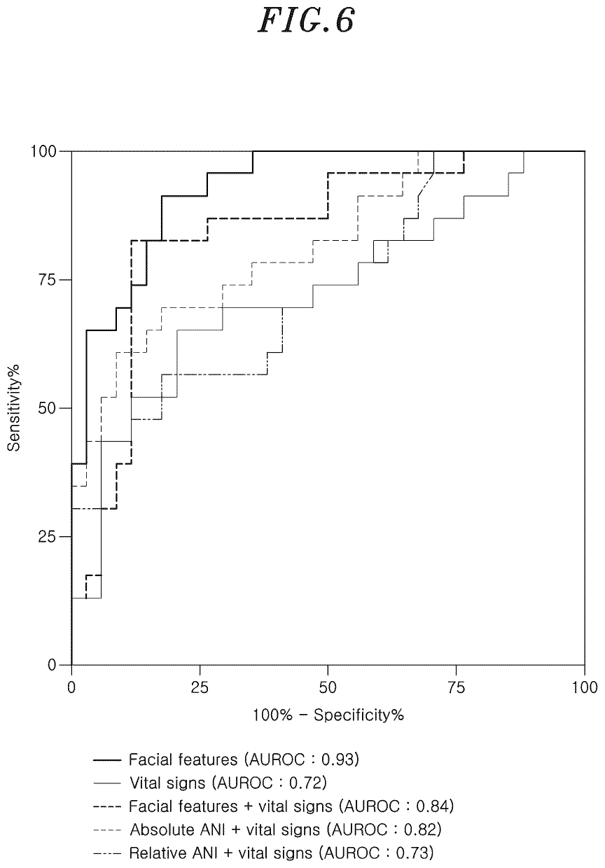

FIG. 6 is a diagram exemplarily illustrating the pain score prediction accuracy determined based on at least one AU representing a facial expression according to an embodiment.

Specifically, FIG. 6 shows the pain score prediction accuracy of an artificial intelligence model trained by considering factors such as at least one AU (facial features), vital signs, and the Analgesia Nociception Index for acute pain patients or post-operative pain patients.

More specifically, the pain score prediction accuracy of the artificial intelligence model trained based on at least one AU is 0.93, the pain score prediction accuracy of the artificial intelligence model trained based on vital signs is 0.72, the pain score prediction accuracy of the artificial intelligence model trained based on at least one AU and vital signs is 0.84, the pain score prediction accuracy of the artificial intelligence model trained based on the absolute Analgesia Nociception Index and vital signs is 0.82, and the pain score prediction accuracy of the artificial intelligence model trained based on the relative Analgesia Nociception Index and vital signs is 0.73.

As such, it can be confirmed that the pain score prediction performance of the artificial intelligence model trained based on at least one AU representing a facial expression is superior to that of an artificial intelligence model trained based on other factors.

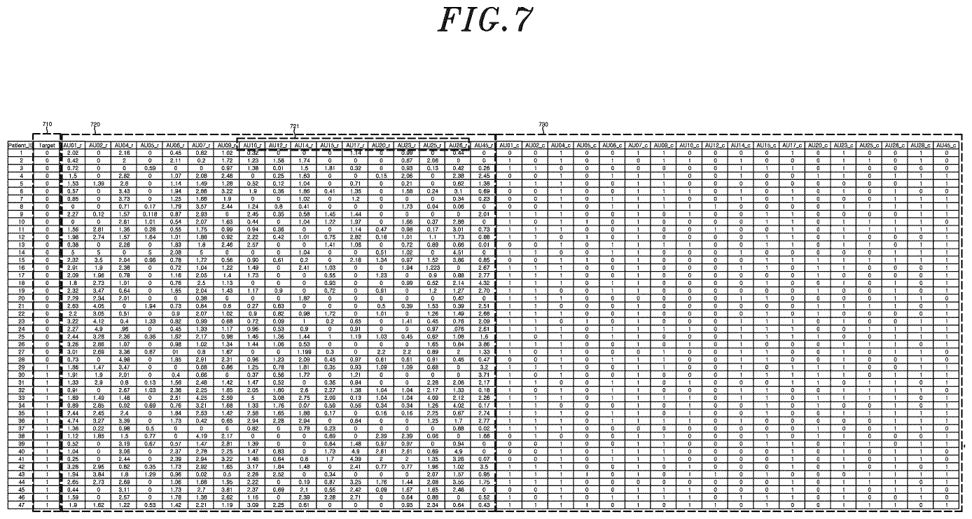

FIG. 7 is a diagram exemplarily illustrating a training dataset of an artificial intelligence model according to an embodiment.

The artificial intelligence model according to an embodiment may be trained to determine the pain score of a target patient by inputting features for at least one AU (720, 730) based on an NRS label (710).

Referring to FIG. 7, the NRS label (710) may refer to a binarized label that classifies a facial image of a target patient (i.e., patient nos. 1 to 47) as 0 (i.e., when NRS is less than 7) or 1 (i.e., when NRS is 7 or more).

Furthermore, the features for the at least one AU may refer to a binarized value (730) that determines whether the facial image corresponds to an AU corresponding to the upper facial region, the lower facial region, the facial direction, or the gaze, or a value related to the intensity of the corresponding AU (720).

Meanwhile, the artificial intelligence model according to an embodiment may be trained to determine the pain score of the target patient based on a first weight assigned to an AU associated with the patient's lip shape (721).

Combinations of steps in each flowchart attached to the present disclosure may be executed by computer program instructions. Since the computer program instructions can be mounted on a processor of a general-purpose computer, a special purpose computer, or other programmable data processing equipment, the instructions executed by the processor of the computer or other programmable data processing equipment create a means for performing the functions described in each step of the flowchart. The computer program instructions can also be stored on a computer-usable or computer-readable storage medium which can be directed to a computer or other programmable data processing equipment to implement a function in a specific manner. Accordingly, the instructions stored on the computer-usable or computer-readable recording medium can also produce an article of manufacture containing an instruction means which performs the functions described in each step of the flowchart. The computer program instructions can also be mounted on a computer or other programmable data processing equipment. Accordingly, a series of operational steps are performed on a computer or other programmable data processing equipment to create a computer-executable process, and it is also possible for instructions to perform a computer or other programmable data processing equipment to provide steps for performing the functions described in each step of the flowchart.

In addition, each step may represent a module, a segment, or a portion of codes which contains one or more executable instructions for executing the specified logical function(s). It should also be noted that in some alternative embodiments, the functions mentioned in the steps may occur out of order. For example, two steps illustrated in succession may in fact be performed substantially simultaneously, or the steps may sometimes be performed in a reverse order depending on the corresponding function.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments. The protection scope of the present disclosure should be interpreted based on the following claims and it should be appreciated that all technical scopes included within a range equivalent thereto are included in the protection scope of the present disclosure.

1. A method for predicting a pain score using a pre-trained artificial intelligence model, the method comprising:
extracting features for at least one action unit (AU) representing a facial expression by inputting a facial image of a target patient; and
determining the pain score of the target patient by inputting the features for the at least one AU representing the facial expression into the pre-trained artificial intelligence model,
wherein the artificial intelligence model is trained to determine the pain score based on a first weight assigned to an AU associated with a patient's lip shape.

2. The method of claim 1, wherein in the extracting of the features for the at least one AU,
the target patient is an acute pain patient or a post-operative pain patient.

3. The method of claim 1, wherein the extracting of the features for the at least one AU comprises:
determining a facial feature point of the target patient using an OpenFace algorithm; and
extracting the features for the at least one AU representing the facial expression based on matching the facial feature point of the target patient and an AU set.

4. The method of claim 3, wherein the extracting of the features for the at least one AU further comprises:
extracting features for a gaze or a facial direction of the target patient.

5. The method of claim 1, wherein the determining of the pain score comprises:
determining the pain score by further inputting features related to a gaze or a facial direction of the target patient into the pre-trained artificial intelligence model.

6. The method of claim 1, wherein the artificial intelligence model is trained to determine the pain score based on a training dataset including a facial image of the patient and a Numeric Rating Scale (NRS) label corresponding to the pain of the patient.

7. The method of claim 1, wherein the artificial intelligence model is further trained to determine the pain score based on a second weight assigned to features related to a gaze of the patient or a third weight assigned to features related to a facial direction of the patient.

8. The method of claim 7, wherein the first weight is higher than the second weight and the third weight.

9. An apparatus for predicting a pain score using a pre-trained artificial intelligence model, the apparatus comprising:
a memory storing a pain score prediction program including one or more instructions; and
a processor that loads the pain score prediction program from the memory and executes the pain score prediction program,
wherein the one or more instructions, when executed by the processor, cause the processor to:
input a facial image of a target patient to extract features for at least one action unit (AU) representing a facial expression, and
input the features for the at least one AU representing the facial expression into the pre-trained artificial intelligence model to determine the pain score of the target patient,
wherein the artificial intelligence model is trained to determine the pain score based on a first weight assigned to an AU associated with a patient's lip shape.

10. The apparatus of claim 9, wherein the target patient is an acute pain patient or a post-operative pain patient.

11. The apparatus of claim 9, wherein the one or more instructions, when executed by the processor, cause the processor to:
determine a facial feature point of the target patient using an OpenFace algorithm, and
extract the features for the at least one AU representing the facial expression based on matching the facial feature point of the target patient and an AU set.

12. The apparatus of claim 11, wherein the one or more instructions, when executed by the processor, cause the processor to extract features for a gaze or a facial direction of the target patient.

13. The apparatus of claim 9, wherein the one or more instructions, when executed by the processor, cause the processor to determine the pain score by further inputting features related to a gaze or a facial direction of the target patient into the pre-trained artificial intelligence model.

14. The apparatus of claim 9, wherein the artificial intelligence model is trained to determine the pain score based on a training dataset including a facial image of the patient and a Numeric Rating Scale (NRS) label corresponding to the pain of the patient.

15. The apparatus of claim 9, wherein the artificial intelligence model is further trained to determine the pain score based on a second weight assigned to features related to a gaze of the patient or a third weight assigned to features related to a facial direction of the patient.

16. The apparatus of claim 15, wherein the first weight is higher than the second weight and the third weight.

17. A computer-readable non-transitory recording medium storing a computer program, wherein the computer program, when executed by a processor, causes the processor to:
extract features for at least one action unit (AU) representing a facial expression by inputting a facial image of a target patient; and
determine the pain score of the target patient by inputting the features for the at least one AU representing a facial expression into a pre-trained artificial intelligence model,
wherein the artificial intelligence model is trained to determine the pain score based on a first weight assigned to an AU associated with a patient's lip shape.

18. (canceled)
